(19) United States
(12) Patent Application Publication
(10) Pub. No.: US 2006/0270925 A1
(43) Pub. Date: Nov. 30, 2006

(54) Tonometer apparatus

(75) Inventors: Jui-Chi Wang, Taipei (TW); Wen-Feng Yang, Hsinchu County (TW); Shiow-Chen Wang, Taipei (TW)

Correspondence Address: BAKER & MCKENZIE LLP, Pennzoil Place, South Tower, 711 Louisiana, Suite 3400, HOUSTON, TX, 77002-2716, US

(73) Assignee: TreatYou Medical Technology Co.

(21) Appl. No.: 11/140,159

(22) Filed: May 27, 2005

(51) Int. Cl. A61B 3/16

(52) U.S. Cl. 600/399

(57) ABSTRACT

A tonometer includes a chamber containing a fluid medium and having a contact tip for contacting an eye surface, the contact tip having a flexible surface stretched by the fluid medium, a pressure sensor detecting a pressure variation of the fluid medium, a detecting mechanism determining whether the tonometer is correctly placed against the eye surface, and a processing circuit configured to treat electrical a signal received from the detecting mechanism.

### FIELD OF THE INVENTION

The present invention generally relates to tonometry measurements, and more particularly to the construction of a tonometer apparatus that can obtain accurate and precise measurement data of the intraoccular pressure.

### DESCRIPTION OF THE RELATED ART

Glaucoma is a disorder that arises when the fluid pressure within an eye, usually called intraoccular pressure, increases to levels that the eye cannot withstand. The result is blindness if such pressure increase is not detected early. Glaucoma can be detected and evaluated via measuring the eye's fluid pressure with a tonometer apparatus.

The general principle of a conventional tonometer applies a force against the eye to depress or flatten its surface, and then measures the amount of force necessary to produce the flattening or depressing. Conventional tonometers can be divided in two types: contact type tonometers and non-contact type tonometers. The mechanism of a conventional non-contact tonometer usually includes applying a puff of compressed air of known volume and pressure against the eye surface, and then detecting via sensor devices the time necessary to achieve a predetermined amount of deformation of the eye surface by application of the air puff. Though the non-contact tonometer can prevent problems such as extraneous pressure on the eye or transmission of disease by contact, it is expensive and requires a skilled manipulation by the operator.

On the other hand, a conventional contact tonometer generally includes a probe end that applies a pressure by contact with the eye surface, and sensor devices that sense movement of the probe end in response to a counterforce produced by the intraoccular pressure. U.S. Pat. Nos. 4,951,671 and 5,165,409 issued to Coan, the disclosure of which is incorporated herein by reference, describe an example of contact tonometer. These two patents describe a contact tonometer which probe end is constructed from a movable shaft slidably mouted to the tonometer housing. When in pressing contact against the eye surface, the probe end slides rearward to deform a membrane of an air chamber and accordingly compresses the air inside the air chamber. An air pressure sensor detects the air pressure variation, which reflects the intraoccular pressure.

The aforementioned contact tonometer of the prior art has some disadvantages. For example, the movement of the probe end produces mechanical frictions that interfere with the measurement data obtained from the pressure sensor. Another disadvantage is that the conventional contact tonometer is incapable of giving an information with respect to the pressing force applied by the operator to urge the probe end against the eye surface; this pressing force may vary each time a measurement is taken, which can result in an imprecision of the obtained measurement data.

Therefore, there is presently a need for a tonometer apparatus that can provide accurate measurement data and inform the operator whether the tonometer is adequately positioned for measurement so that imprecision due to inadequate placement of the tonometer is prevented.

### SUMMARY OF THE INVENTION

In one embodiment, a tonometer apparatus according to the invention comprises a chamber containing a fluid medium and having a contact tip provided with a flexible surface stretched by the fluid medium, a pressure sensor configured to sense a pressure variation of the fluid medium in response to a pressing action exerted by an operator to press the contact tip against an eye surface, a detecting mechanism configured to determine whether the tonometer is correctly placed against the eye surface, and a processing circuit configured to treat electrical signals received from the detecting mechanism and the pressure sensor. In some embodiments, the chamber and the contact tip are formed in a single body. In some embodiments, the chamber includes a flexible portion that resiliently contracts when the contact tip is pressed against the eye surface.

In some embodiments, the detector mechanism includes a movable member configured to slide in a first direction in response to a pressing action exerted by the operator in a second direction to press the contact tip against the eye surface, and a position detector configured to determine whether the movable member sliding in the first direction goes beyond a reference position.

In some embodiments, the position detector includes a light-emitting device and a light-sensing device configured to detect whether light emitted from the light-emitting device travels through a portion of the movable member. In some embodiments, the position detector includes a plurality of springs placed apart from one another, so that a sliding movement of the movable member beyond a predetermined position in the first direction causes a contact between at least two springs indicating that the tonometer is not in the reference position.

In some implementations, the processing circuit emits an error signal in response to a signal from the position detector indicating the movable member has gone beyond the reference position. In variant implementations, the processing circuit obtains measurement data from the pressure sensor when the movable member is at the reference position. In other variations, the processing circuit emits an error signal in response to a signal from the position detector indicating the movable member has left the reference measurement position.

The foregoing is a summary and shall not be construed to limit the scope of the claims. The operations and structures disclosed herein may be implemented in a number of ways, and such changes and modifications may be made without departing from this invention and its broader aspects. Other aspects, inventive features, and advantages of the invention, as defined solely by the claims, are described in the non-limiting detailed description set forth below.

### BRIEF DESCRIPTION OF THE DRAWINGS

FIG. 1A is a cross-sectional view of a tonometer apparatus according to an embodiment of the invention;

FIG. 1B is a cross-sectional view of a tonometer apparatus pushed against an eye surface to measure an intraoccular pressure according to an embodiment of the invention;

FIG. 1C is a cross-sectional view of a tonometer apparatus in an overpressure position according to an embodiment of the invention;

FIG. 2 is a schematic view of a detector position implementation in a tonometer apparatus according to an embodiment of the invention;

FIG. 3 is a circuit diagram illustrating an electric circuit implementation of a tonometer apparatus according to an embodiment of the invention; and

FIG. 4 is a schematic view of a detector position implementation in a tonometer apparatus according to another embodiment of the invention.

### DETAILED DESCRIPTION OF THE EMBODIMENTS

This application describes a tonometer apparatus provided with a position detecting mechanism that can detect any overpressure of the contact tip of the tonometer against the eyeball surface.

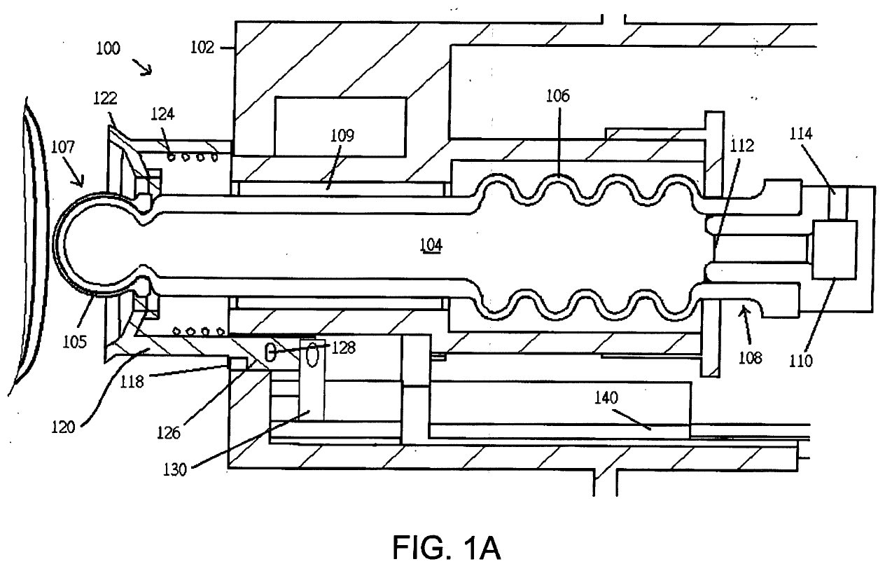

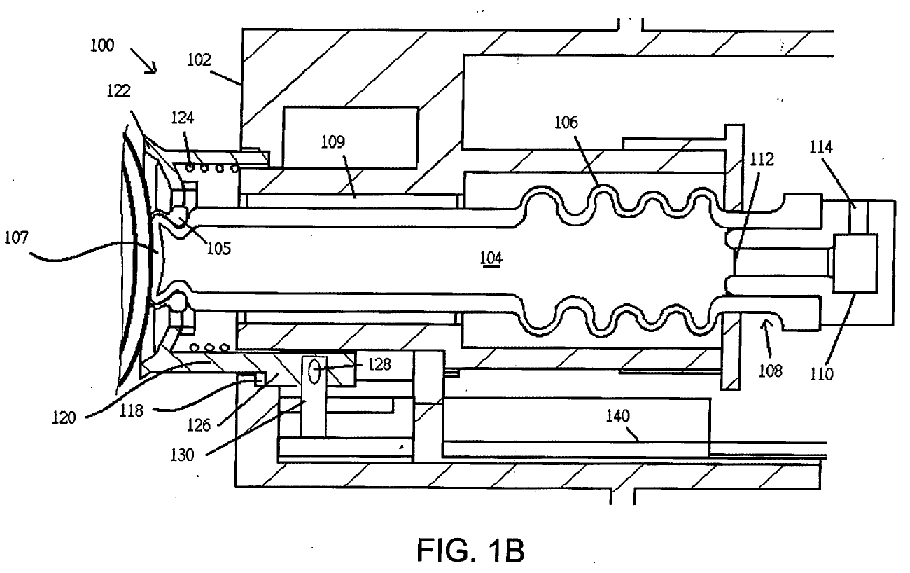

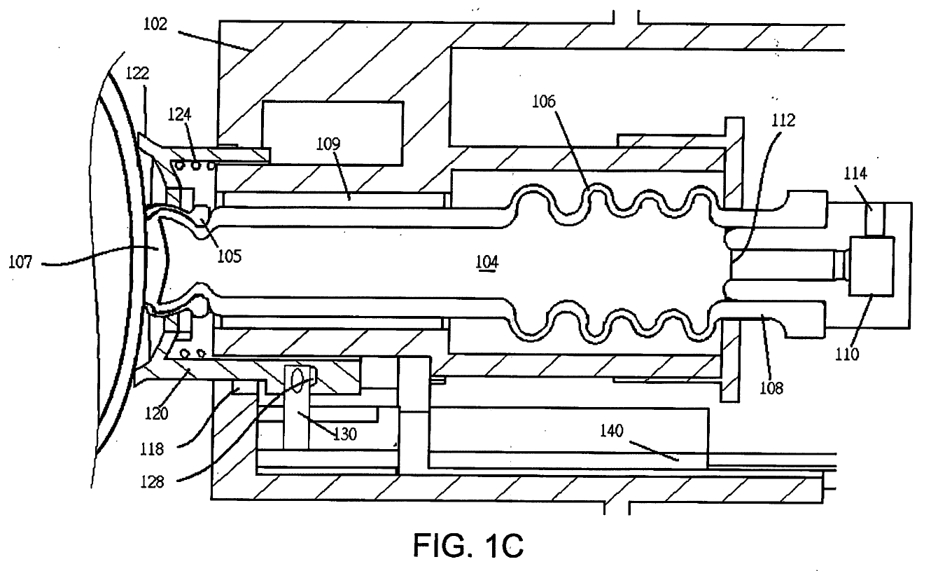

FIG. 1A is a schematic view illustrating the construction of a tonometer apparatus according to an embodiment of the invention. Reference numeral 100 generally designates the tonometer apparatus. The tonometer 100 comprises a case 102 inside which is securely mounted a fluid chamber 104. The fluid chamber 104 is formed in a single elongated hollow body that includes a flexible portion 106 arranged between two opposite ends. Any flexible materials such as elastic rubber may be adequate to form the fluid chamber 104. A first end of the fluid chamber 104 forms a deformable contact tip 107 and a second end 108 of the fluid chamber 104 connects to a pressure sensor 110. In this embodiment, the fluid chamber 104 includes an inner cavity filled with air, which is in contact with an inner side of the contact tip 107. The contact tip 107 thus has a deformable contact surface that is stretched by the air contained within the fluid chamber 104. The elongated body of the fluid chamber 104 lies through a guiding hole 109 of the case 102 and has its contact tip 107 protrude outward through a collar 120. A sheath 105 which may be replaceable or cleanable may cover the contact tip 107 to ensure hygienic contact with the eye surface each time a measurement is taken.

The pressure sensor 110 communicates with the inner cavity of the fluid chamber 104 via a chamber port 112, and has a differential intake port 114 in contact with ambient air. The second end 108 of the fluid chamber 104 forms an elastic mouth that fits and seals over the chamber port 112. The pressure sensor 110 compares the pressure within the fluid chamber 104 via the chamber port 112 with the external ambient pressure via the differential intake port 114.

Referring again to FIG. 1A, the movable collar 120 is slidably mounted to an opening 118 of the case 102, and terminates in a concave cup 122 having a central opening through which the contact tip 107 of the chamber 104 protrudes outwardly. A compressive spring 124 connects the collar 120 to the case 102. The compressive spring 124 exerts a counterforce that causes the collar 120 to slide in a forward direction away from the case 102 once an opposite force pushing the collar 120 rearward toward the inside of the case 102 has been released. The collar 120 includes a rearward portion 126 through which is defined a slot 128.

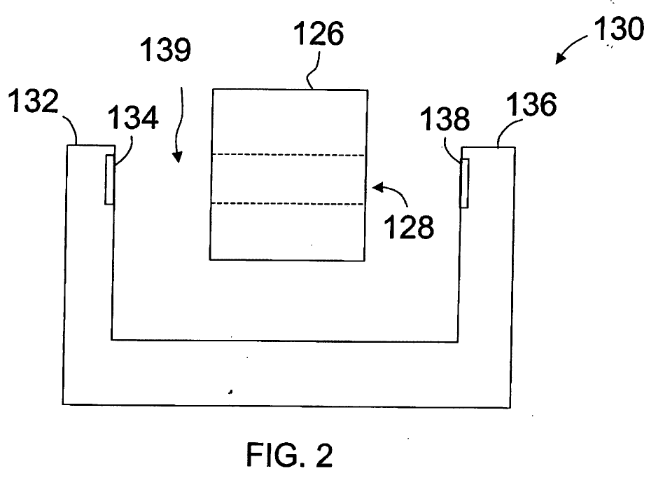

A position detector 130 is mounted adjacent to the rearward portion 126 of the collar 120. As shown in FIG. 2, the position detector 130 has a generally U-shaped body having a first arm 132 provided with a light-emitting diode 134 and a second arm 136 provided with a photo-sensor 138. The rearward portion 126 of the collar 120 passes through a gap 139 between the two arms 132 and 136 so as to selectively obstruct a light path between the light-emitting diode 134 and the photo-sensor 138. When the collar 120 slides to a position where the slot 128 is aligned with the light-emitting diode 134 and the photo-sensor 138, light from the light-emitting diode 134 travels through the slot 128 and strikes on the photo-sensor 138, which accordingly changes the voltage output of the photo-sensor 138. Electric signals outputted from the pressure sensor 110 and the position detector 130 are respectively wired to a circuit board 140 fixedly secured to the case 102.

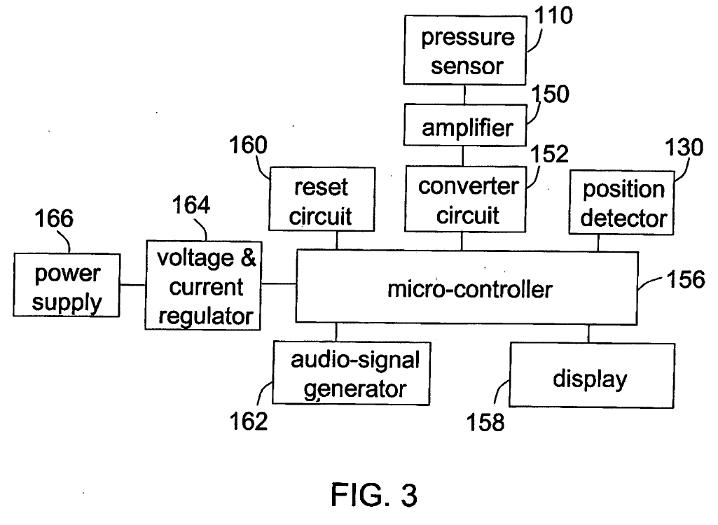

FIG. 3 is a schematic circuit diagram showing a processing circuit implementation of a tonometer apparatus according to an embodiment of the invention. Electric power from a power supply 166 is processed via a voltage and current regulator 164 to supply the electrical circuit with an operating voltage and current. Analog signal outputs from the pressure sensor 110 are converted to suitable digital signals via a converter circuit 152 and sent to a micro-controller 156 for processing. Obtained measurement data are shown on a display 158. A reset circuit 160 is operated to reset the tonometer before each measurement operation. The position detector 130 sends an electric signal to the micro-controller 156 reflecting a position bias with respect to the reference measurement position. The micro-controller 156 includes an inner clock that counts a measurement time period, and determines the number of electric signal changes from the position detector 130 within one measurement time period to determine whether an erroneous manipulation has occurred.

Referring to FIGS. 1A through 1C, the operation of the tonometer apparatus now is described in accordance with an embodiment of the invention. To measure the intraoccular pressure of the eyeball, the contact tip 107 is brought in contact with the eye surface. Then, as the operator moves the tonometer apparatus 100 toward the eye to press the contact tip 107 against the eye surface along a pressing axis of the tonometer, the fluid chamber 104 resiliently retracts rearward by deformation of its flexible portion 106 while the eye surface inwardly deforms. Since the contact tip 107 has a deformable contact surface stretched by the air filled in the chamber 104, damaging contacts to the eye surface are prevented when the operator presses the contact tip 107 against the eye surface.

Referring to FIG. 1B, as the tonometer apparatus 100 moves forward, the cup 122 in turn is urged in contact with the eye surface. Accordingly, the collar 120 slides rearward and compresses the spring 124. When the slot 128 becomes aligned with the position detector 130, the micro-controller 156 detects a change in the output of the photo-sensor 138, which changes from a first voltage level to a second voltage level. The micro-controller 156 accordingly sends a signal to the audio-signal generator 162 to trigger a beep sound informing the operator to hold the reached position for measurement over a time period of about 3 seconds, for example. The micro-controller 156 then obtains measurement data of the intraoccular pressure from the pressure sensor 110. Once the measurement time period lapses, the micro-controller 156 drives the audio-signal generator 162 to emit a second beep sound to inform the operator that the tonometer can be released.

Referring to FIG. 1C, if the slot 128 after being aligned with the position detector 130 slides further rearward along with the rearward movement of the collar 120 before the time period of measurement lapses, the micro-controller 156 detects two voltage level changes from the output of the photo-sensor 138 and accordingly emits two successive beep sounds to inform the operator that an erroneous manipulation of the tonometer 100 has occurred and that the measurement has to be restarted. In the configuration of FIG. 1C, the erroneous manipulation is due to an excessive pressure applied by the operator while pressing the tonometer against the eye surface, which results in a rearward course of the collar 120 that goes beyond the reference measurement position defined by the position detector 130.

On the other hand, if the slot 128 after being aligned with the position detector 130 moves forward and prematurely leaves the reference measurement position before the time period of measurement lapses, the micro-controller 156 also detects two voltage level changes from the output of the photo-sensor 138 and triggers the emission of an error signal. This erroneous manipulation typically corresponds to a premature release of the tonometer by the user during the measurement operation, which causes the collar 120 to slide forward and leave the reference measurement position before the measurement time period lapses.

By defining a reference measurement position along the pressing axis of the tonometer, the operator obtains consistent and reliable intraoccular pressure data because each measurement is obtained with respect to a same reference measurement position of the tonometer. Measurement errors due to a pressing force varying each time a measurement is taken thus are eliminated. In addition, because the contact surface of the contact tip 107 achieves a flexible and deformable interface between the air inside the air chamber and the eye surface, the air pressure sensed by the pressure sensor 110 accurately reflects the intraoccular pressure, and imprecision due to mechanical frictions between movable component parts of the tonometer is eliminated.

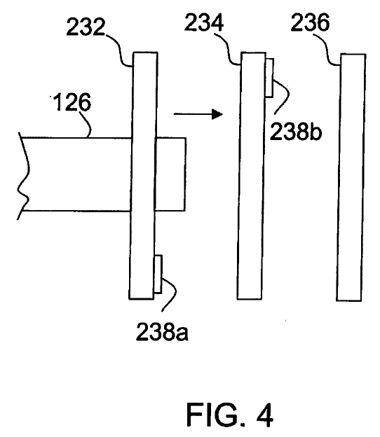

FIG. 4 is a schematic view of a variant mechanism of the position detector implemented in a tonometer apparatus according to another embodiment of the invention. For the sake of simplification, only the mechanism of the position detector is illustrated in FIG. 4. The other parts of the tonometer not illustrated are similar to the embodiments shown in FIGS. 1A through 1C.

In the embodiment shown in FIG. 4, the position detector 230 includes a first spring 232 securely fixed to the rearward portion 126 of the collar, and second and third springs 234, 236 assembled within the case of the tonometer. When the rearward portion 126 of the collar slides rearward in the arrow direction under pressure against the eye surface, the first spring 232 comes in electrical contact with the second spring 234 via contact pad 238a to trigger the measurement. In other words, the reference measurement position is reached when the first and second springs 232, 234 are in electrical contact with each other. If the portion 126 slides further rearward, the second spring 234 in turn is pushed by the portion 126 via the first spring 232 to come in electrical contact with the third spring 236 via contact pad 238b, which accordingly produces a position error signal.

Though two examples have been illustrated, a person skilled in the art of tonometry technique would readily appreciate that many variations of the position detector can be implemented. For example, in another variation not illustrated, the position detector may be implemented through a pressure sensor configured to sense an external force exerted by the operator during pressing of the tonometer toward the eye surface. When the sensed external force exceeds a preset value, the micro-controller can issue an alert signal informing the operator that an excessive pressing action is exerted against the eye surface.

In other variations, the fluid chamber may contain fluid elements other than air chosen with respect to their density so as to provide a pressure variation that adequately reflects the intraoccular pressure during measurement.

Realizations in accordance with the present invention therefore have been described in the context of particular embodiments. These embodiments are meant to be illustrative and not limiting. Many variations, modifications, additions, and improvements are possible. Accordingly, plural instances may be provided for components described herein as a single instance. Structures and functionality presented as discrete components in the exemplary configurations may be implemented as a combined structure or component. These and other variations, modifications, additions, and improvements may fall within the scope of the invention as defined in the claims that follow.

What is claimed is:

1. A tonometer, comprising:
a chamber containing a fluid medium, wherein the chamber includes a flexible contact tip for contacting an eye surface, the contact tip having a flexible surface stretched by the fluid medium;
a pressure sensor configured to sense a pressure variation of the fluid medium;
a detecting mechanism configured to determine whether the tonometer is correctly placed against the eye surface; and
a processing circuit configured to treat a signal received from the detecting mechanism.

2. The tonometer apparatus according to claim 1, wherein the fluid medium includes air.

3. The tonometer apparatus according to claim 1, wherein the chamber and the contact tip are formed in a single body.

4. The tonometer apparatus according to claim 1, wherein the detecting mechanism includes:
a movable member configured to slide in a first direction in response to a pressing action exerted by an operator in a second direction to press the contact tip against the eye surface; and
a position detector configured to determine whether the movable member sliding in the first direction reaches a reference position.

5. The tonometer apparatus according to claim 4, wherein the position detector includes a light-emitting device and a light-sensing device configured to detect whether light emitted from the light-emitting device travels through a portion of the movable member.

6. The tonometer apparatus according to claim 4, wherein the position detector includes a plurality of springs placed apart from one another, so that a sliding movement of the movable member beyond the reference position in the first direction causes a contact between at least two springs indicating that the tonometer is not in the reference position.

7. The tonometer apparatus according to claim 4, wherein the processing circuit emits an error signal in response to a signal from the position detector indicating the movable member has gone beyond the reference position while sliding in the first direction.

8. The tonometer apparatus according to claim 4, wherein the processing circuit obtains measurement data from the pressure sensor when the movable member is at the reference position.

9. The tonometer apparatus according to claim 8, wherein the processing circuit emits an error signal in response to a signal from the position detector indicating the movable member has left the reference position.

10. The tonometer apparatus according to claim 4, wherein the detecting mechanism includes a resilient element configured to resiliently push the movable member in the second direction when the operator releases the pressing action.

11. The tonometer apparatus according to claim 1, wherein the chamber includes a flexible portion that resiliently contracts when the contact tip is pressed against the eye surface.

12. A tonometer apparatus comprising:
a chamber containing a fluid medium, wherein the chamber includes a flexible contact tip having a flexible surface stretched by the fluid medium;
a pressure sensor configured to sense a pressure variation of the fluid medium in response to a pressing action exerted by an operator to press the contact tip against an eye surface;
a detecting mechanism configured to determine an excessive pressing action exerted by the operator against the eye surface; and
a processing circuit configured to treat a signal received from the detecting mechanism.

13. The tonometer apparatus according to claim 12, wherein the fluid medium includes air.

14. The tonometer apparatus according to claim 12, wherein the chamber and the contact tip are formed in a single body.

15. The tonometer apparatus according to claim 12, wherein the detecting mechanism includes:
a movable member configured to slide in a first direction in response to a pressing action exerted by the operator in a second direction to press the contact tip against the eye surface; and
a position detector configured to determine whether the movable member sliding in the first direction goes beyond a reference position indicating an excessive pressing action.

16. The tonometer apparatus according to claim 15, wherein the position detector includes a light-emitting device and a light-sensing device configured to detect whether light emitted from the light-emitting device travels through a portion of the movable member.

17. The tonometer apparatus according to claim 15, wherein the position detector includes a plurality of springs placed apart from one another, so that an excessive pressing action from the operator causes the movable member to slide beyond the reference position in the first direction and cause a contact between at least two springs.

18. The tonometer apparatus according to claim 15, wherein the processing circuit emits an error signal in response to a signal from the position detector indicating the movable member has gone beyond the reference position.

19. The tonometer apparatus according to claim 15, wherein the processing circuit obtains measurement data from the pressure sensor when the movable member is at the reference position.

20. The tonometer apparatus according to claim 19, wherein the processing circuit emits an error signal in response to a signal from the position detector indicating the movable member has left the reference position.

21. The tonometer apparatus according to claim 15, wherein the detecting mechanism includes a resilient element configured to resiliently push the movable member in the second direction when the operator releases the pressing action.

22. The tonometer apparatus according to claim 12, wherein the chamber includes a flexible portion that resiliently contracts when the contact tip is pressed against the eye surface.
